(19) United States
(12) Patent Application Publication
(10) Pub. No.: US 2008/0269648 A1
(43) Pub. Date: Oct. 30, 2008

(54) Method for increasing saliva and tear production with ultrasound

(75) Inventor: Robert T. Bock, Brewster, NY (US)

Correspondence Address: Robert T. Bock, 66 Drovers Lane, Brewster, NY, 10509, US

(73) Assignee: Ultra License Holdings, Inc.

(21) Appl. No.: 11/796,699

(22) Filed: Apr. 30, 2007

(51) Int. Cl. A61N 7/00

(52) U.S. Cl. 601/2

(57) ABSTRACT

A non-invasive apparatus and method for increasing saliva and tear production from the salivary and lacrimal glands with the application of high frequency low intensity ultrasound. An ultrasound transducer coated with connecting gel placed in contact with the facial tissues transmitting 1.5 MHz frequency ultrasound pressure waves into the tissues and into the glands. The time-averaged intensity of the ultrasound pressure waves is typically limited to approximately 30 milliwatts per square centimeter to avoid tissue heating.

### 1. FIELD OF INVENTION

This invention relates to the increase of the production of saliva and tears by the human body, and more particularly their increase by the application of ultrasound.

### 2. DESCRIPTION OF PRIOR ART

Dry mouth and dry eyes are serious and often painful diseases caused by inadequate production of saliva by the salivary glands and tears by the lacrimal glands. If not treated successfully, dry mouth causes sores or split skin at the corners of the mouth, cracked lips, sore throat, and gum and teeth disease. The inadequate production and maintenance of the tear film in the eye causes stinging and burning sensation in the eyes, eye fatigue, difficulty of wearing contact lenses, and blurred vision.

In the past decades numerous attempts has been made to increase saliva production by various medications and by electrical means. Systemic parotid stimulants such as parasympathomimetics agonists were prescribed; artificial saliva and oral lubricants used; and electro-stimulation including transcutaneous electric nerve stimulation were applied with various and limited success and detrimental side effects.

The side effects of the parasympathomimetics agonists such as the increased sweating, diarrhea, hypertension, hypotension, and tachycardia restrict the use of these products; transcutaneous electric nerve stimulation (TENS) devices are capable of increasing parotid saliva flow, however patients are experiencing side effects such as twitching of facial musculature and anesthesia of cutaneous areas adjacent to the placement of the device.

Treatments of dry eyes are the application of artificial tears, cyclosporine, and plugging of the tear ducts to conserve tears. When people experience intolerable irritation from dry eyes despite the frequent use of lubricating eye-drops, doctors may prescribe steroid drops. Long term use of cyclosporine and steroids both come with detrimental side effects therefore their application is limited.

What has occurred to date is that not withstanding the teachings of the prior art, the ability to increase saliva and tear production effectively, inexpensively, and easily has remained unsolved.

### 3. OBJECTS AND SUMMARY OF THE INVENTION

Responding to the above described unresolved needs; the object of this invention is to increase saliva and tear production effectively, inexpensively, and easily without detrimental side effects. Application of ultrasound energy in a totally non-invasive way is possible by applying a piezo electric transducer to the facial anatomy at the location of the salivary and lacrimal glands. The application is non invasive because it uses high frequency low intensity ultrasound typical in the ultrasonic imaging applications. This method while being absolutely safe, also avoids the side effects of medications and transcutaneous electric nerve stimulation.

The treatment with ultrasound is highly effective. Increased saliva output was observed and measured by saliva collection cups. While we have proven clinically the effectiveness of this ultrasonic method to increase salivary gland activity, the exact underlying mechanism of how the ultrasound pressure waves stimulate gland activity is yet to be determined. We believe that as the ultrasound pressure waves propagate through the tissues, it applies high frequency mechanical forces causing compression and rarefaction of the fluids and cells. These ultrasonic frequency mechanical forces may cause vibrations of the cell membrane or the extracellular matrix. The high frequency vibration of the membrane may alter ionic permeability and therefore second-messenger activity, which initiates the biological response.

### 4. BRIEF DESCRIPTION OF THE DRAWINGS

In the accompanying drawings, in which certain modes of carrying out the present invention are shown for illustrative purposes:

FIG. 1 is a schematic of the embodiment of the invention.

FIG. 2 depicts the application of the ultrasonic energy to the salivary gland.

FIG. 3 depicts the application of the ultrasonic energy to the lacrimal gland.

### 5. DESCRIPTION OF THE PREFERRED EMBODIMENTS

Referring in detail to the drawings, the reference numerals herein refer to the like numbered parts in the drawings. In the following discussion, unless otherwise qualified, the term “ultrasound” refers to either continuous wave ultrasound or a repetitive burst type ultrasonic modality.

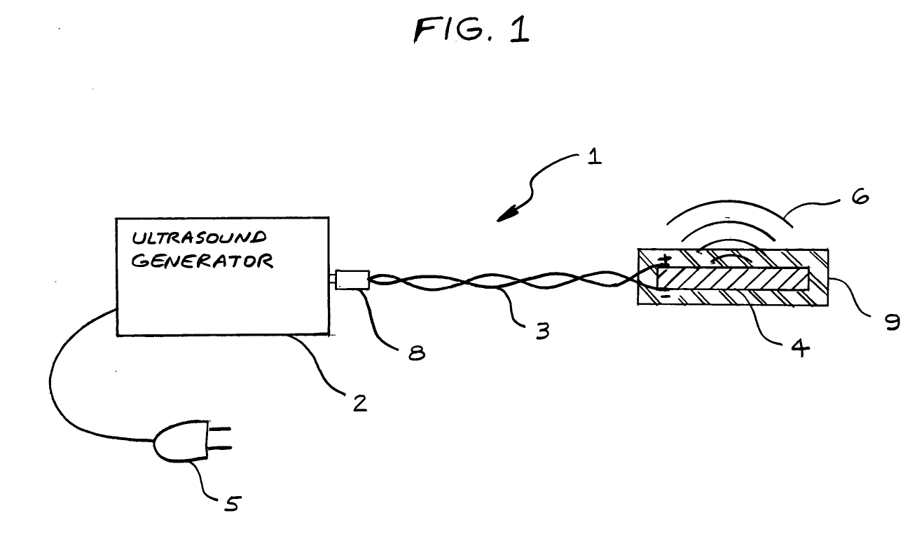

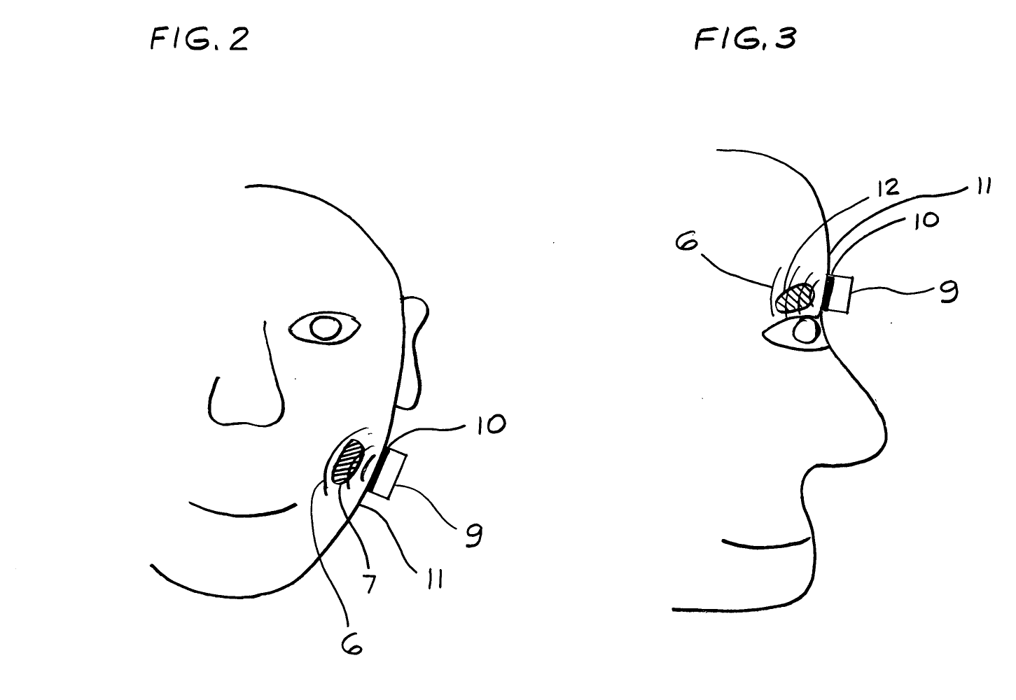

The saliva output-increasing method and device 1, in accordance with the simplest form of the present invention, is shown in FIG. 1 and FIG. 2 and FIG. 3. The device 1 comprises of a piezo-electric crystal 4, connecting wiring 3 and connector 8 that connects the piezo-electric crystal 4 to the ultrasound generator 2. The piezo-electric crystal 4 is typically a PZT-8 or similar material and constructed by well-known methods utilized in the ultrasound imaging art. The ultrasound generator 2 draws its power either from a standard household current through connector 5 or is operated from a battery within the generator. The piezo-electric crystal 4 is encapsulated in a plastic material forming a sealed transducer 9. The piezo-electric crystal 4 contracts and expands in tune with the ultrasonic frequency signals supplied by the ultrasound generator 2 to generate ultrasonic pressure waves 6 which are coupled to the facial tissues 11. The transmission of the pressure waves 6 into the facial tissues 11 can be enhanced by the application of a common ultrasound coupling gel 10 or simply by Vaseline. The ultrasonic pressure waves 6 propagate through the facial tissues into the salivary gland 7 to increase saliva production. Transducer 9 is held in place by a bandage or simply by the fingers of the user during the treatment period. Alternatively, transducer 9 is placed on the facial tissues 11 in front of the lacrimal gland 12 to irradiate the lacrimal gland 12 with ultrasonic pressure waves 6 to increase tear production.

The intensity of the ultrasonic pressure waves 6 is ideally limited to be approximately 30 mW/cm², which is effective for the purpose and at the same time it is below the tissue heating range. However, higher intensities can be applied with the appropriate safeguards against tissue heating or damage. The ultrasonic pressure waves 6 could be applied in a continuous wave modality or in a pulsed burse mode such as 200-microsecond burse width repeated at 1 kilohertz repetition rate to further limit tissue heating. The ideal frequency of the ultrasonic pressure waves 6 is approximately 1.5 MHz, but it can range from 20,000 Hertz to above 5,000,000 Hertz.

While the preceding description contain many specificities, these should not be construed as limitations on the scope of the invention, but rather as an exemplification of a preferred embodiment. Many other variations are possible. Skilled artisans will readily be able to change dimensions, shapes and construction materials of the various components described in the embodiment, and apply ultrasound to other bodily fluid producing glands within the anatomy. Accordingly, the scope of the invention should be determined not by the embodiment illustrated, but by the appended claims and their legal equivalents.

I claim:

1. A method for increasing saliva production in a patient comprising the step of applying ultrasound pressure waves to the salivary glands through the facial tissue of the patient.

2. A method as defined in claim 1 wherein the ultrasound pressure waves having a frequency in the range of 1-2 MHz, and intensity in the range of 100-200 milliwatts per square centimeter.

3. A method as defined in claim 2 wherein said ultrasound pressure waves are applied in 200 microseconds bursts repeated in 1 kilohertz intervals.

4. A method for increasing tear production in a patient comprising the step of applying ultrasound pressure waves to the lacrimal glands through the facial tissue of the patient.

5. A method as defined in claim 4 wherein the ultrasound pressure waves having a frequency in the range of 1-2 MHz, and intensity in the range of 100-200 milliwatts per square centimeter.

6. A method as defined in claim 5 wherein said ultrasound pressure waves are applied in 200 microseconds bursts repeated in 1 kilohertz intervals.

7. A method for increasing bodily fluid productions in a patient comprising the step of applying ultrasound pressure waves to the glands producing said bodily fluids.
